(19) United States
(12) Patent Application Publication
(10) Pub. No.: US 2023/0357725 A1
(43) Pub. Date: Nov. 9, 2023

(54) LIVER MICROTISSUE AND USES IN TREATMENT OF LIVER FAILURE

(71) Applicant: TREEFROG THERAPEUTICS, PESSAC (FR)

(72) Inventors: Maxime FEYEUX, TALENCE (FR); Dilyana Aleksandrova TODOROVA, Pessac (FR); Elise laure WARTER, TALENCE (FR)

(21) Appl. No.: 18/072,191

(22) Filed: Nov. 30, 2022

(30) Foreign Application Priority Data
May 6, 2022 (FR) FR2204315

(51) Int. Cl. C12N 5/071; A61K 35/407; A61K 35/413; A61L 27/38; A61L 27/36

(52) U.S. Cl. CPC C12N 5/0671; C12N 5/0697; A61K 35/407; A61K 35/413; A61L 27/3886; A61L 27/3804; A61L 27/3604; C12N 2506/45; C12N 2513/00; A61L 2430/28

(57) ABSTRACT

The invention relates to a liver microtissue, preferably, the liver microtissue having the largest dimension between 500 and 700 μm, and the liver microtissue expressing CYP3A4 monooxygenase with an activity of at least 75,000 RLU per million cells and producing at least 18 ag of urea per million cells per 24 hours. The invention also relates to a method for producing the liver microtissue from pluripotent stem cells, and uses of the liver microtissue in treatment and/or prevention of liver failure.

### CROSS-REFERENCE TO RELATED APPLICATIONS

This application claims the benefit of priority from French patent application FR 2204315 filed May 6, 2022, the entire disclosure of which is herein incorporated by reference in its entirety.

### TECHNICAL FIELD

The present invention relates to the treatment of liver failure by the use of liver microtissue obtained from bioengineered cell microcompartments. The invention relates in particular to a liver microtissue produced from pluripotent stem cells, its preparation methods and uses.

### BACKGROUND

The liver is one of the most complex organs in the human body. An integral part of the digestive system, the liver is constantly supplied with nutritious or toxic substances resulting from digestion. The processing of these substances by the liver is essential for the body and has the following objectives:
storage and distribution of nutrients from digestion
degradation of toxic substances
synthesis of most blood proteins, and
bile production.

To perform these functions, the liver is composed of a wide variety of cells, such as hepatocytes, bile duct cells (cholangiocytes), stellate cells (Ito cells), Kupffer cells, mesenchymal stem cells and endothelial cells.

Hepatocytes are the most represented liver cells and are responsible for the majority of liver functions, from fatty acid synthesis to urea production to plasma protein synthesis.

Cholangiocytes are the polarized epithelial cells forming the walls of the bile ducts. They have a role in regulating the secretion of bile and in collecting it in order to transport it from the hepatocytes to the intestine.

The other cells provide good vascularization as well as signaling functions and interactions with other liver cells and with cells of the immune system.

When the liver can no longer perform its functions, it is called liver failure. The main causes of liver failure are viral infections, drug overdoses, immunological disorders, hereditary diseases or blood circulation disorders. When damage to liver function is irreversible, the recommended treatment is liver transplantation.

Thus, liver transplantation represents the standard treatment for people with end-stage liver disease.

Today, there are great difficulties in finding organ donors who can supply a liver of sufficient quality for a transplant.

Like any organ transplant, liver transplantation can only be performed if the liver is of good quality in order to avoid the risks associated with the transplant such as infections, cancers, prolonged immunosuppression and major surgery.

Today, only ⅔ of patients can benefit from a transplant and only when their quality of life has greatly deteriorated. In addition, the cost to be expected for a liver transplant is around €1 million (or $1 million) in the United States.

Faced with these problems, several innovative solutions have emerged.

Transplantation of isolated hepatocytes has appeared as an attractive approach, but clinical trials have remained few and inconclusive, limited by the poor survival, integration and expansion of isolated hepatocytes following graft in vivo, factors therefore limiting the short- and long-term therapeutic effects. In fact, hepatocytes isolated from donors have a very limited capacity for proliferation in vivo and in vitro. Moreover, the isolated hepatocytes put in culture tend to enter a process of dedifferentiation, thus decreasing the chances of obtaining a sufficient number of mature hepatocytes.

Although this technique makes it possible to treat a large number of patients, the administered dose is very often insufficient and presents a risk during the injection of the cells escaping into the general circulation.

This is mainly due to the difficulty for single cells to integrate within the liver, as well as poor quality induced by the preparation of the primary cells and their culture in vitro and the rejection of part of the hepatocytes despite immunosuppression.

Consequently, the low number of hepatocyte donors, the stability and the limited functionality of these hepatocytes constitute a barrier to their use

The development of protocols for the guided differentiation of pluripotent stem cells, that is to say, embryonic stem cells and induced pluripotent stem cells, has however made it possible to obtain an almost inexhaustible source of hepatocytes.

Although promising, hepatocytes derived from pluripotent cells are very difficult to cultivate on a large scale and generate disproportionate production costs. For example, the expected cost for the production of autologous liver grafts from induced pluripotent stem cells is around 9.7 million dollars.

To date, guided differentiation protocols do not make it possible to produce a variety of functional cell phenotypes, and in particular enough mature hepatocytes; the cells obtained retain the characteristics of fetal liver hepatocytes, in particular with a persistent expression of alpha-fetoprotein and low albumin production.

Nevertheless, certain protocols make it possible to obtain liver cells with better functional characteristics. These protocols remain complex and require steps of dissociation, reaggregation or co-cultures with in particular mesenchymal stem cells and endothelial cells. These additional steps add additional risks, increasing the total cost and decreasing the control of the finished product.

For an application in cell therapy, it is necessary to be able to adapt the existing methods in order to: i) obtain production with a limited number of steps in order to be effective on a large scale, ii) improve the integration of the grafted cells. The techniques developed today do not make it possible to produce liver microtissue obtained from induced pluripotent stem cells suitable for large-scale culture and presenting functional hepatocytes.

The current methods for producing liver microtissue are still too complex, have multiple steps and therefore a very high cost, and are difficult to scale up.

There is therefore a significant need for a solution allowing large-scale production of liver microtissue comprising several phenotypes of liver cells, which can be produced on a large scale and can be directly transplanted, to meet an essential demand for liver grafts.

The object of the invention is therefore to meet all of these needs and to overcome the drawbacks and limitations of the prior art.

### SUMMARY OF THE INVENTION

To meet this objective, the invention proposes a bioengineered liver microtissue, suitable for uses in cell therapy and in particular in treating liver failure.

For this purpose, the subject of the invention is a three-dimensional liver microtissue, the largest dimension of which is between 500 and 700 μm, expressing CYP3A4 monooxygenase with an activity of at least 75,000 RLU per million cells and producing at least 18 μg of urea per million cells per 24 hours.

Advantageously, the activity of the CYP3A4 associated with urea production makes it possible to guarantee a liver microtissue comprising at least functional liver cells.

Preferably, the liver cells secrete at least 75 μg of albumin per million cells per 24 hours.

Thus, the liver microtissue exhibits metabolic activity similar to a healthy liver.

According to another object, the invention relates to a liver microtissue comprising at least 3 different phenotypes of liver cells, said cells of the microtissue all having been obtained from induced pluripotent stem cells encapsulated in a closed three-dimensional microcompartment.

Advantageously, the liver microtissue has sufficient cellular diversity to sustainably restore and/or improve liver function.

Preferably, the liver microtissue comprises at least immature hepatocytes, mature hepatocytes and cholangiocytes.

According to a preferred object of the invention, the liver microtissue comprises at least:
liver cells, of which at least 40% of the liver cells are cells expressing cytokeratin 19 (CK19), and
cells expressing CD73 and CD90.

Preferably, the cells expressing CD73 and CD90 are mesenchymal stem cells and the cells expressing CK19 are cholangiocytes.

Advantageously, the phenotypic composition of the liver microtissue is close to that of a healthy human liver.

Preferably, the liver microtissue according to the invention comprises:
at least one lumen,
at least one cell of the microtissue in contact both with a lumen and with the medium outside the microtissue, and
at least one cell surrounded only by cells.

Advantageously, the organization of the liver microtissue is close to the organization of the liver tissue in development, which enables it in particular to promote its integration into the liver and the proper performance of the metabolic functions in the treated liver. At the same time, the liver microtissue is a bioengineered tissue and it has the same genotype as pluripotent stem cells from which cells in the liver microtissue are a progeny.

According to a particularly preferred embodiment, the liver microtissue is in an ovoid, cylindrical, spheroid or spherical or substantially ovoid, cylindrical, spheroid or spherical or ellipsoidal shape. Preferably, the liver microtissue is in an ellipsoidal shape or in substantially ellipsoidal shape.

Advantageously, the ellipsoidal shape of the microtissue makes it possible to promote the survival of the liver microtissue. Thus, a greater part of the liver microtissues is integrated by the treated liver.

Preferably, the liver microtissue according to the invention comprises at least one bile duct and/or at least one glycogen granule.

According to a preferred object of the invention, the liver microtissue comprises:
between 50 and 99% liver cells, including between 20 and 60% cells expressing CK19, and
between 1 and 20% cells expressing CD73 and CD90.

An object of the invention is also a set of several three-dimensional liver microtissues in a medium, of which at least one liver microtissue is a liver microtissue according to the invention. Preferably, at least 50% (by number) of the liver microtissues of the set of liver microtissues are liver microtissues according to the invention.

According to another aspect, the invention relates to a closed three-dimensional cell microcompartment comprising an outer hydrogel layer defining an inner part, said inner part comprising at least one liver microtissue according to the invention.

The microcompartment according to the invention provides a microenvironment suitable for the culture of pluripotent stem cells and for their differentiation into cells constituting the microtissue according to the invention. Indeed, such a microcompartment makes it possible to reproduce in vitro the in vivo conditions of the cellular microenvironment during liver organogenesis.

The invention also relates to a set of cell microcompartments according to the invention.

According to another aspect, the invention relates to a method for preparing a microcompartment or a set of microcompartments comprising at least the implementation of the following steps: producing a microcompartment comprising induced pluripotent stem cells and inducing cell differentiation within the microcompartment so as to obtain at least 3 different phenotypes of liver cells.

In yet another aspect, the invention relates to a liver microtissue according to the invention or a microcompartment containing it, or a set of liver microtissues according to the invention or a set of microcompartments containing them, for its use as a drug, preferably in the prevention or treatment of liver failure caused by diseases such as hepatic fibrosis and cirrhosis, steatosis, non-alcoholic steatosis, hepatitis, metabolic diseases of the liver, diseases related to secretion of factors VIII, alpha 1 antitrypsin, IX and/or VWF, Wilson's disease and hereditary hemochromatosis.

Within the meaning of the invention, methods of treatment and/or prevention are equivalent to the uses described in this disclosure.

In yet another aspect, this invention relates to a drug comprising the three-dimensional liver microtissue according to this invention or a closed three-dimensional cell microcompartment comprising an outer hydrogel layer defining an inner part, said inner part comprising the microtissue.

In yet further aspect, this invention relates to a method of treating symptoms associated with liver failure in a subject, the method comprising administering to the subject the three-dimensional liver microtissue according to this invention, or a closed three-dimensional cell microcompartment comprising an outer hydrogel layer defining an inner part, said inner part comprising the microtissue. In particular, methods include embodiments wherein the liver failure is acute, chronic or acute-on-chronic liver failure.

In yet another aspect, this invention relates to a method of treating or preventing a metabolic disease of the liver in a subject, the method comprising administering to the subject the three-dimensional liver microtissue according to this invention, or a closed three-dimensional cell microcompartment comprising an outer hydrogel layer defining an inner part, said inner part comprising the microtissue.

In yet further aspect, this invention relates to a method of treating or preventing in a subject hepatic fibrosis and cirrhosis, steatosis, non-alcoholic steatosis, hepatitis, a disease linked to secretion of factor VIII and factor IX and VWF, Wilson's disease, or hereditary hemochromatosis, the method comprising administering to the subject the microtissue according to this invention, or an closed three-dimensional cell microcompartment comprising an outer hydrogel layer defining an inner part, said inner part comprising the microtissue.

In yet another aspect, this invention relates to a method for preparing the microcompartment according to this invention, the method comprising the steps consisting in:
a) producing a closed three-dimensional cell microcompartment comprising induced pluripotent stem cells inside an outer hydrogel layer, and optionally comprising extracellular matrix elements or a natural or synthetic extracellular matrix, and
b) inducing cell differentiation within the cell microcompartment, so as to obtain a microcompartment comprising liver cells. In some preferred embodiments of the method, step b) may last at least 20 days. Some preferred embodiments of the method include those wherein between 40 and 150 induced pluripotent stem cells are present in the microcompartment in step a).

In yet another aspect, this invention relates to a method for preparing the liver microtissue according to this invention, wherein the method comprises:
preparing a closed three-dimensional cell microcompartment comprising an outer hydrogel layer defining an inner part, said inner part comprising the liver microtissue, and
eliminating the outer hydrogel layer to recover the liver microtissue.

In yet another aspect, this invention relates to a method for transplanting a liver tissue into a subject, the method comprising transplanting to the subject one or more of the three-dimensional liver microtissues of this invention and/or a closed three-dimensional cell microcompartment comprising an outer hydrogel layer defining an inner part, said inner part comprising the microtissue. Some embodiments of the method include those, wherein the subject is at risk of liver failure and/or has been diagnosed with a metabolic disease of the liver.

### BRIEF DESCRIPTION OF THE FIGURES

FIG. 1a is a comparative representation of the expression of the genes of interest (EOMES, CXCR4, HHEX, PROX1, AFP, ASGR1) during differentiation according to the differentiation protocol.

FIG. 1b is a comparative representation of the expression of the genes of interest (SOX17, FOXA2, TBX, HNF4A, ALB, KT18) during differentiation according to the differentiation protocol.

FIG. 1c is a comparative representation of the expression of the genes of interest (GATA4, HNF1B, SOX9, KT19, TAT) during differentiation according to the differentiation protocol.

FIG. 2 is a comparative representation of the expression of the genes of interest (EOMES, CXCR4, FOXA2, SOX17) according to the culture conditions.

FIG. 3 is a comparative representation of the expression of proteins of interest (SOX17, FOXA2) depending on the culture conditions, 5 days after the start of differentiation.

FIG. 4a is a comparative representation of the expression of the genes of interest (PROX1, TBX, AFP, KT18, KT19, HNF4A) according to the culture conditions.

FIG. 4b is a comparative representation of the expression of the genes of interest (HNF1B, SOX9, ASGR1, ALB, TAT)) according to the culture conditions.

FIG. 5 is a comparative representation of expansion factor for 30 days as a function of the culture conditions.

FIG. 6 is a graphic representation of albumin secretion during differentiation as a function of culture conditions.

FIG. 7 is a graphical representation of urea production during differentiation as a function of culture conditions.

FIG. 8 is a graphical representation of CYP3A4 activity as a function of culture conditions.

FIG. 9 is a set of images obtained by confocal microscopy of a microcompartment presenting hepatocytes.

FIG. 10a is a set of images obtained by confocal microscopy of a microcompartment from D-0 to D-9 after the start of differentiation.

FIG. 10b is a set of images obtained by confocal microscopy of a microcompartment from D-12 to D-30 after the start of differentiation.

FIG. 10c is a set of images obtained by confocal microscopy of a microcompartment on D-15 after the start of differentiation.

FIG. 10d is a set of images obtained by confocal microscopy of a microcompartment on D-20 after the start of differentiation.

FIG. 10e is a set of images obtained by confocal microscopy of a microcompartment on D-30 after the start of differentiation.

FIG. 11 is a set of images obtained by confocal microscopy of a microcompartment identifying certain liver cell phenotypes.

### DETAILED DESCRIPTION OF THE INVENTION

### Definitions

Within the meaning of the invention, “alginate” means linear polysaccharides formed from β-D-mannuronate and α-L-guluronate, salts and derivatives thereof.

Within the meaning of the invention, “hydrogel capsule” or “hydrogel microcompartment” means a three-dimensional structure formed from a matrix of polymer chains, swollen with a liquid and preferably water.

Within the meaning of the invention, “human cells” means human cells or immunologically humanized non-human mammalian cells. Even when this is not specified, the cells, stem cells, progenitor cells and tissues according to the invention consist of or are obtained from human cells or from immunologically humanized non-human mammalian cells.

Within the meaning of the invention, “embryonic stem cell” means a pluripotent stem cell of a cell derived from the internal cell mass of the blastocyst. The pluripotency of embryonic stem cells can be assessed by the presence of markers such as transcription factors OCT4, NANOG and SOX2 and surface markers such as SSEA3/4, Tra-1-60 and Tra-1-81. The embryonic stem cells used in the context of the invention are obtained without destroying the embryo from which they originate, for example using the technique described in Chang et al. (Cell Stem Cell, 2008, 2(2)): 113-117). Alternatively, human embryonic stem cells may be excluded.

Within the meaning of the invention, “a cell surrounded only by cells” in a microtissue means a cell that is neither in contact with a lumen nor in contact with the outside of the microtissue.

Within the meaning of the invention, “mutant cell” means a cell carrying at least one mutation.

Within the meaning of the invention, “pluripotent stem cell” or “pluripotent cell” means a cell that has the capacity to form all the tissues present in the entire organism of origin, but without being able to form an entire organism as such. Human pluripotent stem cells may be referred to as hPSCs in this application. They may in particular be induced pluripotent stem cells (iPSC or hiPSC for human induced pluripotent stem cells), embryonic stem cells or MUSE (“Multilineage-differentiating Stress Enduring”) cells.

Within the meaning of the invention, “induced pluripotent stem cell” means a pluripotent stem cell induced to pluripotency by genetic reprogramming of differentiated somatic cells. These cells are notably positive for pluripotency markers, such as alkaline phosphatase staining and expression of NANOG, SOX2, OCT4 and SSEA3/4 proteins. Examples of methods for obtaining induced pluripotent stem cells are described in the articles by Yu et al. (Science 2007, 318 (5858): 1917-1920), Takahashi et al. (Cell, 207, 131(5): 861-872) and Nakagawa et al. (Nat Biotechnol, 2008, 26(1): 101-106).

Within the meaning of the invention, “progenitor cell” means a stem cell already engaged in the differentiation into liver cells but not yet differentiated.

Within the meaning of the invention, methods of treatment and/or prevention are equivalent to the uses described this disclosure. “A method of treatment and/or prevention” is not restricted to a complete cure. Instead, “a method of treatment and/or prevention” includes any method comprising steps which may lead to amelioration of at least some of the symptoms at least temporarily, improve or sustain the quality of life at least for a period of time and/or delay or decrease the worthening of at least some of the symptoms for at least a period of time. “A method” is a treatment and prevention method if a subject reports an improvement in at least some of his symptoms and/or the subject does not report an onset of new symptoms or the subject does not report worthening of at least some of the symptoms.

Within the meaning of the invention, “a liver cell obtained from an induced pluripotent stem cell” means that the liver cell originates from the induced pluripotent stem cell and the liver cell is a progeny cell of the pluripotent stem cell either directly or through several rounds of division and differentiation of progenitor cells off-springing from the induced pluripotent stem cell.

Within the meaning of the invention, “Feret diameter” of a microcompartment (or part of a microcompartment) or of a microtissue according to the invention means the distance “d” between two tangents to said microcompartment (or to said part) or of the microtissue, these two tangents being parallel, such that the entire projection of said microcompartment (or of said part) or of the microtissue lies between these two parallel tangents. A Ferret diameter of the inner part of the microcompartment is measured between two interfaces of the inner part and of the outer layer of the microcompartment, that is to say, the distance “d” between two tangents to said inner part, these two tangents being parallel, such that the entire projection of said inner part is comprised between these two parallel tangents.

Within the meaning of the invention, “variable thickness” of a layer means the fact that the layer for the same microcompartment or the same microtissue does not have the same thickness everywhere.

Within the meaning of the invention, “microcompartment” or “capsule” means a partially or completely closed three-dimensional structure, containing several cells.

Within the meaning of the invention, “microtissue” or “liver microtissue” means a three-dimensional human tissue comprising at least liver cells and whose largest dimension is less than 1 mm.

Within the meaning of the invention, “medium” means an aqueous solution including cells or microtissues, compatible with the survival, development and/or metabolism of the cells. It may be a culture medium.

Within the meaning of the invention, “convective culture medium” means a culture medium animated by internal movements.

Within the meaning of the invention, “mutation” means a genetic or epigenetic mutation, preferably a functional mutation. It may in particular be a specific modification of the genetic sequence, a structural variant, an epigenetic modification, or a modification of the mitochondrial DNA.

Within the meaning of the invention, “functional mutation” means a transmissible genetic or epigenetic modification that confers a gain or loss of function or potential loss of function on the mutant cell concerned. It is preferably a mutation resulting in a modification of the phenotype of the mutant cell concerned. Very preferably, it is a change in the sequence of the genome and/or the epigenome that alters the therapeutic potential of a population of cells, either by increasing the risk associated with the therapy produced or by reducing the benefit provided by the therapy produced.

Within the meaning of the invention, “smallest dimension” of a microcompartment or of a layer of cells means the value of the smallest Feret diameter of said microcompartment.

Within the meaning of the invention, “light” or “lumen” means a volume of aqueous solution topologically surrounded by cells. Preferably, its content is not in diffusive equilibrium with the volume of convective liquid present outside the microcompartment.

According to the invention, “smallest radius” of the inner part of a microcompartment means half the value of the smallest Ferret diameter of the inner part of the microcompartment.

According to the invention, “average radius” of the inner part of a microcompartment means the average of the radii of the smallest compartment, each radius corresponding to half the value of a Ferret diameter of the inner part of the microcompartment.

According to the invention, “rate of expansion at X days” means a measurement of cell proliferation at time t=X. The rate of expansion is measured by taking the ratio of the number of cells counted on day X of the culture divided by the number of cells at the start of the culture (day of encapsulation or day of placement in culture).

According to the invention, “large-scale culture” means a cell culture method suitable for a production batch of liver microtissue making it possible to treat at least 1 patient, preferably 10 patients, more preferably 100 patients, even more preferably more than 1,000 patients.

According to the invention, “functional phenotype” of a microtissue means the presence of mature hepatocytes characterized by the expression of albumin and the absence of expression of alpha-fetoprotein and cytokeratin 19.

According to the invention, “liver bud” means a cellular organization characterized by a cellular extension of the endoderm of the embryonic foregut that gives rise to the parenchyma of the liver and the bile duct. This is a particular conformation giving rise to hepatocytes and cholangiocytes.

According to the invention, “RLU” means relative light units. It corresponds to the measurement of the quantity of light produced by a bioluminescence reaction. This measurement can be carried out using a luminometer.

Liver Microtissue

The object of the invention is therefore a three-dimensional liver microtissue, the largest dimension of which is between 500 and 700 μm, expressing CYP3A4 monooxygenase with an activity of at least 75,000 RLU per million cells and producing at least 18 μg of urea per million cells per 24 hours. The three-dimensional liver microtissue is produced by a biogenetic method which includes culturing and differentiating pluripotent stem cells in a microcompartment.

Cytochromes P450 are hemoproteins participating in the oxidative metabolism of many molecules. Cytochromes P450 are enzymes involved in the biotransformation of exogenous compounds, both in the phenomena of detoxification and intoxication by formation of reactive entities. The most abundant human hepatic form (CYP3A4) is responsible for the metabolism of more than 60% of drugs. Its presence within the microcompartment is a functional guarantee. Urea is a nitrogenous product resulting from the catabolism of proteins. It is exclusively synthesized in the liver via the urea cycle and the amount of urea formed depends on the amount of protein ingested, protein catabolism and the state of liver function. A CYP3A4 activity of at least 75,000 RLU per million cells associated with a urea production of at least 18 μg per million cells per 24 hours guarantees the presence of mature hepatocytes as well as sufficient metabolic activity to guarantee a significant effect on impaired liver functions.

Preferably, the activity of CYP3A4 is at least 80,000 RLU, even more preferably at least 100,000 RLU.

According to a preferred embodiment, the liver microtissue produces at least 40 μg of urea per million cells per 24 hours, even more preferably at least 60 μg, in particular at least 80 μg

Advantageously, the liver microtissue according to the invention exhibits a metabolic activity close to that of a healthy liver. Thus, liver microtissue is particularly effective in restoring the functions of the treated liver.

Preferably, the liver microtissue according to the invention comprises between 50 and 99% liver cells.

The liver microtissue according to the invention can be obtained from induced pluripotent stem cells.

According to a variant, the liver microtissue can be obtained from stem cells, progenitor cells and/or cells capable of differentiating into liver cells.

Preferably, the liver microtissue is obtained from induced pluripotent stem cells encapsulated in a single closed three-dimensional microcompartment.

The liver microtissue preferentially comprises liver cells secreting at least 75 μg of albumin per million cells per 24 hours, even more preferentially at least 150 μg, in particular at least 250 μg.

According to a particular embodiment, the liver microtissue is obtained from induced pluripotent stem cells and comprises liver cells secreting at least 75 μg of albumin per million cells per 24 hours at least 20 days after the start of differentiation.

Albumin is the most abundant protein in the blood. Produced by the liver, albumin is in particular responsible for stabilizing blood pressure and transporting many substances.

Advantageously, an albumin production of at least 75 μg per million cells per 24 hours is an indicator of proper functioning of the liver microtissue.

According to another preferred embodiment, the liver microtissue comprises at least 3 different phenotypes of liver cells, all the cells of the microtissue all having been obtained from induced pluripotent stem cells encapsulated in a single closed three-dimensional microcompartment.

Preferably, the liver microtissue comprises at least 4 different phenotypes of liver cells, even more preferably at least 5.

Advantageously, the liver microtissue according to the invention has a significant cellular diversity making it possible to reproduce the liver microenvironment. The diversity and their proximity to the cells present in the liver microtissue allows a large number of cellular interactions thus cooperating in the performance of numerous metabolic and transport functions.

The cellular diversity found in the liver microtissue according to the invention is obtained from induced pluripotent stem cells in a single closed three-dimensional microcompartment. In this way, the different cell types can organize themselves within the microcompartment reproducing the liver microenvironment.

According to a preferred embodiment, all the cells of the microtissue have all been obtained by differentiation of induced pluripotent stem cells encapsulated in a single closed three-dimensional microcompartment, said induced pluripotent stem cells preferably all being of the same line.

Very preferably, all the cells of the microtissue have all been obtained from induced pluripotent stem cells encapsulated in a single enclose three-dimensional microcompartment by a single differentiation method implemented in said microcompartment.

Preferably, the induced pluripotent stem cells before differentiation in microtissue according to the invention form a cyst in the microcompartment. Thus, preferably, all the cells of the microtissue have all been obtained from at least one cyst of induced pluripotent stem cells encapsulated in a single closed three-dimensional microcompartment, preferably by differentiation, in particular by a single differentiation method implemented in said microcompartment.

Advantageously, the microenvironment of the microcompartment from which the microtissue according to the invention is obtained reproduces the conditions of liver organogenesis. Indeed, the microcompartment according to the invention makes it possible to limit the various physical and/or stress constraints and promotes the interactions between the various cell types within the microcompartment.

According to a particularly suitable embodiment, the liver microtissue according to the invention comprises at least immature hepatocytes, mature hepatocytes and cholangiocytes. Preferably, it comprises at least:
immature hepatocytes, characterized by the expression of alpha-fetoprotein (AFP) and albumin (ALB) and the absence of expression of cytokeratin 19 (CK19)
mature hepatocytes, characterized by the expression of albumin and the absence of expression of alpha-fetoprotein and cytokeratin 19
cholangiocytes, characterized by the expression of cytokeratin 19 and the absence of expression of albumin and alpha-fetoprotein.

According to a variant, the liver microtissue according to the invention comprises hepatoblasts. Hepatoblasts can be characterized by the expression of alpha-fetoprotein, albumin and cytokeratin 19.

The presence of these liver cell phenotypes warrants a multifactorial effect on liver functions. This effect may restore or optimize certain liver functions.

In a particular embodiment, the liver microtissue comprises at least 50% mature and/or immature hepatocytes.

According to a particularly suitable embodiment, the liver microtissue comprises, within the liver cells, between 20 and 60% (by number) cells expressing cytokeratin 19.

Cells expressing cytokeratin 19 are preferentially cholangiocytes.

In the context of the invention, the liver cells are preferably chosen from mature hepatocytes, immature hepatocytes, hepatoblasts, cholangiocytes and mixtures thereof.

Liver microtissue may also comprise cells expressing CD73 and CD90. Cells expressing CD73 and CD90 are preferentially mesenchymal stem cells.

Mesenchymal stem cells are a cell population well known for its properties on tissue repair and regeneration, particularly of the liver. Advantageously, the presence of mesenchymal stem cells derived from induced pluripotent stem cells of the patient to be treated makes it possible to guarantee efficacy of the tissue repair of the liver exhibiting liver failure.

Thus, in a particular embodiment, the microtissue according to the invention comprises at least:
liver cells, preferably at least immature hepatocytes, mature hepatocytes, cholangiocytes, and
cells expressing CD73 and CD90, preferably at least mesenchymal stem cells.

Preferably, the liver microtissue comprises at least:
liver cells, of which between 20 and 60% (by number) of the cells are cells expressing cytokeratin 19, and
cells expressing CD73 and CD90.

The liver microtissue preferably comprises (percentages by number):
between 50 and 99% liver cells, preferably of which between 20 and 60% (by number) of the cells are cells expressing cytokeratin 19, and
between 1 and 20% cells expressing CD73 and CD90.

The liver microtissue according to the invention can also comprise other cells, such as in particular Ito cells (stellate cells), Kupffer cells, endothelial cells, hepatoblasts. Thus, in another particular embodiment, the liver microtissue comprises:
mature hepatocytes, immature hepatocytes, hepatoblasts, cholangiocytes,
Ito cells and/or Kupffer cells and/or endothelial cells,
and possibly mesenchymal stem cells.

According to a variant, the liver microtissue according to the invention can also comprise smooth muscle cells and/or fibroblasts.

The cell phenotypes comprised in the liver microtissue are preferably compatible with the liver microenvironment.

The cellular diversity offered by the liver microtissue according to the invention makes it possible to repair and regenerate the diseased liver so as to restore the affected functions.

The concentration of mature hepatocytes, cholangiocytes and mesenchymal stem cells ensures a lasting effect during cell therapy. It is particularly important when culturing to obtain a content of mature and/or immature hepatocytes greater than 50% in order to obtain a sufficient effect as soon as possible after microtissue graft.

A concentration of mature and/or immature hepatocytes of less than 50% would limit the efficiency of the graft and would require a graft volume that is all the greater as the concentration (by number) of hepatocytes is low. Indeed, it is estimated that at least 5% of the mass of the liver in functional hepatocytes is necessary to treat acute liver failure, for example, and metabolic deficiencies of the liver such as diseases linked to the secretion of factor VIII, factor IX and VWF, Wilson's disease and hereditary hemochromatosis require the same order of magnitude.

Mature hepatocytes express albumin but do not express alpha-fetoprotein. These markers are easily identifiable and quantifiable by detection methods well known to those skilled in the art, such as flow cytometry.

Particularly preferably, the liver microtissue comprises at least one lumen, at least one cell of the microtissue in contact both with a lumen and with the medium outside the microtissue, and at least one cell surrounded only by cells.

In the context of the invention, such an organization is characteristic of a functional microtissue ready to be used in cell therapy.

The particular organization of the microtissue is possible in particular owing to the presence of polarized liver cells making it possible to structure and organize the microtissue.

Thus, the microtissue may contain polarized liver cells. Polarization of liver cells in the microcompartment may be an indicator of functional microtissue formation.

The liver microtissue according to the invention may be in the form of an ellipsoid.

Advantageously, the ellipsoidal shape of the microtissue makes it possible to promote the survival and integration of the liver microtissue. When the liver microtissue is injected into the general circulation, its ellipsoidal shape allows it to facilitate its flow into the blood vessels in the case of administration by injection via the vascular route (in an embodiment via the portal vein), but also to facilitate its flow within a cannula if the administration is done by intra-tissue graft. The improvement in the injection of the liver microtissue causes an improvement in the integration of the liver microtissue by the liver of the treated patient.

The liver microtissue according to the invention preferably has a diameter or a smaller dimension of between 100 and 300 μm, preferably between 150 and 280 μm. The largest dimension of the liver microtissue is preferably less than 1 mm, and is very preferably between 500 and 700 μm.

Advantageously, the size of the liver microtissue is adapted for administration by the portal vein.

The liver microtissue according to the invention may comprise between 300 and 14,000 cells, preferably between 500 and 8,000, even more preferably between 900 and 5,000, in particular 4,500 cells.

Preferably, the liver microtissue comprises at least one bile duct. The bile ducts collect the bile produced by the liver cells to transport it to the gallbladder. The presence of at least one bile duct within the liver microtissue promotes the proper functioning of the microtissue within the liver and the reconstruction of defective bile ducts.

According to another embodiment, the liver microtissue comprises at least one glycogen granule. Glycogen serves as storage for carbohydrates in the body; it is mainly stored in the liver. Under the action of insulin, liver cells store glucose in the form of glycogen. Under the action of glucagon, liver cells will hydrolyze glycogen and release glucose into the blood. The presence of glycogen granules in the liver cell is a physiological element of the functioning of its metabolism and therefore of its function. Thus, the presence of at least one glycogen granule in the liver microtissue guarantees favorable conditions for the functioning of the liver microtissues for an optimal therapeutic effect.

Preferably, the liver microtissue comprises at least one bile duct and at least one glycogen granule.

The liver microtissue according to the invention can be obtained by differentiation of induced pluripotent stem cells at least 20 days, preferably at least 30 days after encapsulation in an enclose three-dimensional microcompartment of 1 to 200 induced pluripotent stem cells, preferably between 5 and 150, especially between 15 and 80.

Preferably, the liver microtissue can be obtained from the encapsulation in a closed three-dimensional microcompartment of 1 to 200 induced pluripotent stem cells, preferably between 5 and 150, in particular between 15 and 80.

Preferably, the induced pluripotent strains form a cyst in the microcompartment before differentiation into cells of the liver microtissue according to the invention.

An object of the invention is also a set of liver microtissues comprising at least one liver microtissue according to the invention. Preferably, it is a set of several three-dimensional liver microtissues in a medium, of which at least one liver microtissue is a liver microtissue according to the invention.

According to a variant, at least 50% (by number) of the liver microtissues of the set of liver microtissues are liver microtissues according to the invention.

The set of liver microtissues according to the invention is adapted to be administered to patients with liver failure. Administration can be by injection in a biocompatible solution. The injection can be carried out in the portal vein so as to reach the liver without dispersion in the general circulation, directly in the liver or ectopically. When the injection is ectopic, it can be carried out in the abdomen or under the renal capsule.

Preferably, the effective quantity of the set of microtissues injected into the patient corresponds to a mass between 1 and 20% of the mass of the liver of the treated patient, preferably between 2 and 10%, in particular 5%.

According to another aspect, the object of the invention is a microcompartment comprising at least one liver microtissue according to the invention.

The microcompartment according to the invention comprises an outer hydrogel layer. Preferably, the hydrogel used is biocompatible, that is to say, it is not toxic to the cells. The outer hydrogel layer must allow the diffusion of oxygen and nutrients to supply the cells contained in the microcompartment and allow their survival. According to one embodiment, the outer hydrogel layer comprises at least alginate. It may consist exclusively of alginate. The alginate may in particular be a sodium alginate, composed of 80% α-L-guluronate and 20% β-D-mannuronate, with an average molecular mass of 100 to 400 kDa and a total concentration of between 0.5 and 5% by mass. The outer hydrogel layer is cell-free.

The outer hydrogel layer makes it possible in particular to protect the cells from the mechanical stress of bioreactors, to limit potentially toxic molecules when accumulated in the medium.

The average outer layer thickness can be variable. It is preferably between 20 and 60 μm, more preferably between 30 and 40 μm. The ratio between the smallest radius of the inner part and this thickness is preferably between 2 and 10 μm.

The microcompartment according to the invention advantageously exhibits a rate of expansion of the induced pluripotent stem cells of at least 15 times, 20 days after the start of differentiation.

The invention thus promotes amplification with a high rate of expansion, which consequently reduces the culture time to obtain a functional microtissue.

The microcompartment according to the invention can be obtained after encapsulation of induced pluripotent stem cells with or without addition of extracellular, natural or synthetic matrix.

According to a variant, the microcompartment according to the invention comprises, in its inner part, extracellular matrix such as Matrigel® and/or Geltrex® and/or a hydrogel-type matrix of plant origin such as modified alginates or of synthetic origin or a copolymer of poly(N-isopropylacrylamide) and poly(ethylene glycol) (PNIPAAm-PEG) of the Mebiol® type.

According to a variant, the microcompartment according to the invention can be obtained after encapsulation of induced pluripotent stem cells without addition of extracellular matrix. The extracellular matrix elements can be peptide or peptidomimetic sequences, mixtures of proteins, extracellular compounds or structural proteins, such as collagen, laminins, entactin, vitronectin, as well as growth factors or cytokines.

Within the microcompartment, the induced pluripotent stem cells differentiate into liver tissue for a period of at least 20 days, preferably at least 30 days.

Unexpectedly, the microcompartment provides a favorable microenvironment for the development of liver microtissue. Indeed, during the differentiation within the microcompartment, the cells organize themselves to form a structure similar to a liver bud. This liver bud is found during liver organogenesis in vivo; it is this particular structure that gives rise to hepatocytes and cholangiocytes. The presence of a structure similar to a liver bud during the formation of the liver microtissue is an indicator of the good quality of the tissue.

Thus, the liver microtissue according to the invention is preferably obtained after the formation of a structure similar to a liver bud in a single closed three-dimensional microcompartment.

The microcompartment according to the invention makes it possible to isolate the induced pluripotent stem cells from the mechanical stresses present within the bioreactor. This mechanical isolation allows the microtissue to set up and maintain a topology that approximates the spatial and structural organization of the liver organogenesis existing in vivo.

Unexpectedly, after at least 20 days, preferably 30 days, of differentiation within the microcompartment, the cells retain their conformation, thus making it possible to have better proliferation while maintaining a functional phenotype. Consequently, this makes it possible to reduce the number of passages and to reduce the time in culture necessary to reach the final number of liver cells required.

The cells present in the microcompartment according to the invention were preferentially obtained after at least two cell division cycles after the encapsulation in an outer hydrogel layer of 1 to 200 induced pluripotent stem cells, preferably between 5 and 150, in particular between 15 and 80.

Preferably, the cells present in the microcompartment according to the invention have been obtained after at least 2, 3, 4, 5, 6, 7, 8, 9, 10, 11, 12, 13, 14, 15, 20, 25, 28, 30 cell division cycles after encapsulation in an outer hydrogel layer, preferably at least 5, even more preferably at least 6, to obtain up to 8,000 mature hepatocytes per microcompartment. For example, mature hepatocytes present in the microcompartment were obtained after at least six cell division cycles after encapsulation of cells in the outer hydrogel layer.

Preferably, the number of cell divisions for implementing the method according to the invention is less than 100, even more preferably less than 30.

Preferably, the microcompartment is obtained after at least 2 passes after encapsulation, more preferably at least 3, 4 or 5 passes. Each pass can for example last between 2 and 10 days, in particular between 2 and 4 days.

Preferably, the microcompartment is obtained after at least one re-encapsulation, more preferably between 1 and 14 re-encapsulations, in particular between 2 and 7 re-encapsulations. Very preferably, a re-encapsulation corresponds to a new pass and each encapsulation cycle corresponds to a pass.

Preferably, the microcompartment according to the invention was harvested in less than 30 days after encapsulation, even more preferably in less than 20 days after encapsulation of at least 1 induced pluripotent stem cell in the inner part defined by the outer hydrogel layer, preferably 5, 20 and up to 100.

The microcompartment according to the invention can contain between 100 and 14,000 cells, preferably between 300 and 10,000 cells, even more preferably between 300 and 5,000, more particularly at least 50 mature hepatocytes and at least 20 cholangiocytes.

Advantageously, the microcompartment according to the invention protects the induced pluripotent stem cells from mechanical stress, thus making it possible to obtain a rate of expansion that is particularly suited to large-scale culture.

The microcompartment according to the invention can be in any three-dimensional form, that is to say, it can have the shape of any object in space. The microcompartment can have any shape compatible with cell encapsulation. Preferably, the microcompartment according to the invention is in a spherical or elongated or ellipsoidal or substantially spherical or elongated shape. It may have the shape of an ovoid, a cylinder, a spheroid or a sphere, or substantially this shape.

It is the outer layer of the microcompartment, that is to say, the hydrogel layer, that gives the microcompartment according to the invention its size and shape. Preferably, the smallest dimension of the microcompartment according to the invention is between 150 μm and 500 μm, preferably between 200 μm and 450 μm.

Its largest dimension is preferably greater than 350 μm, more preferably between 350 μm and 600 μm.

The microcompartment according to the invention can optionally be frozen in order to be stored. It should then preferably be thawed before use.

The invention also relates to several microcompartments according to the invention used together.

The invention also relates to a set or series of microcompartments comprising at least two three-dimensional cell microcompartments, characterized in that at least one microcompartment is a microcompartment according to the invention.

Preferably, the series of microcompartments according to the invention is in a culture medium, in particular in an at least partially convective culture medium. Any culture medium suitable for culturing induced pluripotent stem cells can be used, such as for example the medium “HCM Hepatocyte Culture Medium BulletKit (Lonza)” or “Medium E from William (Thermofisher Scientific),” since the concentration of dissolved salts is compatible with the maintenance of alginate crosslinking by divalent cations.

According to a particularly suitable embodiment, the object of the invention is a series of cell microcompartments in a closed enclosure, such as a bioreactor, preferably in a culture medium in a closed enclosure, such as a bioreactor. Thus, preferably, the microcompartments are placed in a culture medium in a closed bioreactor.

The set or series of microcompartments according to the invention preferably comprises between 2 and 1,016 microcompartments.

Thus, the microcompartment according to the invention is suitable for large-scale culture by providing liver microtissues exhibiting a functional phenotype, in particular vis-à-vis their detoxifying (particularly the activity of CYP3A) and secretory (particularly the secretion of albumin) capacities, guaranteeing optimal efficacy in vivo.

Use of Liver Microtissue According to the Invention

The liver microtissue or the microcompartment according to the invention can be used for all applications, in particular as a drug, in particular in cell therapy in humans.

Thus, the object of the invention is a liver microtissue according to the invention or a set of liver microtissues comprising at least one liver microtissue according to the invention, or a microcompartment according to the invention or a set of microcompartments comprising at least one microcompartment according to the invention, for its use as a drug, in particular in cell therapy.

Preferably, the liver microtissue or the microcompartment according to the invention can be used in the prevention or treatment of symptoms associated with liver failure, in particular acute, chronic or acute-on-chronic liver failure.

Preferably, the liver microtissue or the microcompartment according to the invention can be used in the prevention or treatment of disease such as fibrosis, cirrhosis, steatosis, non-alcoholic steatosis, hepatitis, metabolic diseases of the liver such as diseases linked to secretion of factor VIII and factor IX and VWF, Wilson's disease and hereditary hemochromatosis. Indeed, all these indications can be treated by liver transplantation, and this is precisely what the transplantation of a set of microtissues according to the invention restoring the liver functions is called upon to solve.

Thus, the object of the invention is a liver microtissue according to the invention or a set of liver microtissues comprising at least one liver microtissue according to the invention, or a microcompartment according to the invention or a set of microcompartments comprising at least one microcompartment according to the invention, for its use in the prevention or treatment of symptoms associated with liver failure, in particular acute, chronic or acute-on-chronic liver failure, in particular in the prevention or treatment of liver disease such as fibrosis, cirrhosis, steatosis, non-alcoholic steatosis, hepatitis, metabolic diseases of the liver such as diseases linked to secretion of factor VIII and factor IX and VWF.

The invention also relates to a liver microtissue according to the invention or a set of liver microtissues comprising at least one liver microtissue according to the invention, or a microcompartment according to the invention or a set of microcompartments comprising at least one microcompartment according to the invention, for its use in the evaluation of molecules or in the modeling of liver diseases.

Method for Preparing Microcompartments and Microtissues According to the Invention

The invention also relates to a method for preparing microcompartments according to the invention.

The method for preparing a microcompartment or a set of microcompartments according to the invention may comprise the following steps:
(a) preparing a closed three-dimensional cell microcompartment comprising, inside an outer hydrogel layer, induced pluripotent stem cells, and optionally elements of extracellular matrices or a natural or synthetic extracellular matrix,
(b) inducing cell differentiation within the cell microcompartment, so as to obtain a microcompartment comprising liver cells.

The preparation of the microcompartment of step (a) can be carried out in any culture medium suitable for culturing induced pluripotent stem cells such as mTeSR™1 or mTeSRPlus media from Stemcell technologies, StemMACS™ iPS-Brew XF (Miltenyi Biotec or StemFlex from thermofisher Scientific).

The microcompartment of step (a) can be obtained by encapsulation of 1 to 150 induced pluripotent stem cells, preferably at least 50, in particular at least 100.

Preferably, the encapsulation is implemented according to techniques known to those skilled in the art. Indeed, any method of producing cell microcompartments containing inside an outer hydrogel layer and cells can be used to implement the preparation method according to the invention. In particular, it is possible to prepare microcompartments by adapting the method and the microfluidic device described in Alessandri et al., 2016 (“A 3D printed microfluidic device for production of functionalized hydrogel microcapsules for culture and differentiation of human Neuronal Stem Cells (hNSC),” Lab on a Chip, 2016, vol. 16, no. 9, pp. 1593-1604), following the steps described below.

Preferably, step (a) is implemented by adding extracellular matrix elements or a natural or synthetic extracellular matrix. According to a variant, step (a) is implemented without adding extracellular matrix elements or extracellular matrices or a natural or synthetic extracellular matrix.

In the context of the invention, step (a) is preferably implemented in a device capable of generating hydrogel capsules using a microfluidic chip. For example, the device can comprise syringe pumps for several solutions injected concentrically using a microfluidic injector that makes it possible to form a jet that splits into drops that are then collected in a calcium bath. According to a particularly suitable embodiment, two or three solutions loaded onto two or three syringe pumps:
a hydrogel solution, for example alginate,
optionally, an isotonic intermediate solution, preferably an isotonic solution not containing a divalent cation such as Ca2+Mg2+ to avoid crosslinking of the hydrogel too early in the injector, such as for example a sorbitol solution,
a solution comprising induced pluripotent stem cells and culture medium

The three solutions are co-injected (injected simultaneously) concentrically using a microfluidic injector or microfluidic chip that makes it possible to form a jet that splits into drops, the outer layer of which is the hydrogel solution and the core of which is the solution of step (a) comprising the induced pluripotent stem cells; these drops are collected in a calcium bath that crosslinks and/or gels the alginate solution to form the shell.

To improve the mono-dispersity of the cell microcompartments, the hydrogel solution is preferably charged with a direct current at (between 1 and 10 kV). A grounded ring may optionally be placed at a distance from the tip of between 1 mm and 20 cm, preferably 3 mm to 10 cm, even more preferably 1 cm to 5 cm, from the tip in the plane perpendicular to the axis of the jet emerging from the microfluidic injector (coextrusion chip) to generate the electric field.

According to the invention, it is necessary to generate capsules whose inner part has an average radius or smaller radius of at least 100 μm. To generate capsules with such dimensions with a coextrusion chip (microfluidic injector or microfluidic chip), the invention proposes in particular to modify the flow rate of the coextruded solutions and the final opening of the coextrusion chip. “Flow rate” means the flow rate of each solution that arrives at the injector. “Final opening of the co-extrusion chip” means the inner opening of the output channel of the chip.
for the flow rate: we preferably go from a value between 20 and 40 mL/h for each coextruded solution for the standard sizes of capsules known from the prior art, to a value between 45 and 150 mL/h, preferably between 45 and 110 mL/hour for a variant of the invention,
for the diameter of the final opening of the coextrusion chip (microfluidic injector), which goes from a value preferably between 50 and 120 μm for the standard sizes of capsules known from the prior art, to a diameter value comprised between 150 and 300 μm, preferably between 180 and 240 μm.

Thus, according to a particular embodiment, the encapsulation of step (a) is carried out using a microfluidic injector whose final opening diameter is between 150 and 300 μm, preferably between 180 and 240 μm, and with the flow rate of each of the 3 solutions comprised between 45 and 150 mL/h, preferably between 45 and 110 mL/h.

According to a variant, step (a) of preparing a microcompartment can be carried out with stem cells, progenitor stem cells or cells capable of differentiating into liver cells.

Step (b) of cell differentiation is preferably carried out for at least 20 days, even more preferably at least 30 days.

During the differentiation of step (b), the cells organize themselves in an organization similar to that of a liver bud. This organization is typically marked by the structuring in the form of two cell sub-populations presenting two different organizations, one of the epithelial type (i.e. in the form of apicobasally polarized cell base and presenting tight junctions and the other of the mesenchymal type, that is to say, without apicobasal organization and presenting focal junctions.

The differentiation of the induced pluripotent stem cells of step (b) into liver microtissue can be carried out by any known differentiation method, as described by Raggi et al., Stem cell Report 2022 or Mallanna and Duncan, Curr Protoc Cell Bil, 2014.

According to one embodiment, steps (a) and/or (b) are implemented with permanent or sequential stirring. This stirring is important because it maintains the homogeneity of the culture environment and avoids the formation of any diffusive gradient.

Preferably, step (b) is carried out under hypoxia conditions; more preferably, the first 5 days of differentiations are carried out under hypoxia conditions.

Unexpectedly, differentiation under hypoxia conditions allows a better rate of expansion to be obtained.

Implementing the method according to the invention makes it possible to obtain microcompartments comprising at least 100, preferably at least 500, at least 800, at least 1,000, in particular at least 3,000 cells.

The method according to the invention is preferably implemented in a closed enclosure such as a closed bioreactor.

The invention also relates to a method for preparing a liver microtissue comprising implementing the following steps:
(a) preparing a closed three-dimensional cell microcompartment comprising, inside an outer hydrogel layer, induced pluripotent stem cells, and optionally extracellular matrix elements or a natural or synthetic extracellular matrix,
(b) inducing cell differentiation within the cell microcompartment so as to differentiate the induced pluripotent stem cells into liver cells
(c) removing the outer hydrogel layer to recover liver microtissue of ovoid, cylindrical, spheroid or spherical or approximately ovoid, cylindrical, spheroid or spherical or ellipsoid shape comprising at least 300 cells expressing CYP3A4 monooxygenase with an activity of at least 75,000 RLU per million cells and producing at least 18 μg of urea per million cells per 24 hours

Step (c) consists in dissociating the microcompartment to obtain liver microtissue; the removal of the outer hydrogel layer can be carried out in particular by hydrolysis, dissolution, piercing and/or rupture by any means that is biocompatible, that is to say, non-toxic for the cells. For example, removal can be achieved using phosphate buffered saline, a divalent ion chelator, an enzyme such as alginate lyase if the hydrogel comprises alginate, and/or laser microdissection.

Step (c) of removing the outer hydrogel layer makes it possible to recover the liver microtissue of ovoid, cylindrical, spheroid or spherical or substantially ovoid, cylindrical, spheroid or spherical or ellipsoidal shape comprising at least 300 cells corresponding to at least 3 different liver cell phenotypes.

Preferably, the liver microtissue recovered in step (c) has an ellipsoidal shape.

The liver microtissue from step (c) may comprise at least 300, in particular between 300 and 14,000, even more preferably between 900 and 5,000 cells.

The liver microtissue from step (c) comprises at least one lumen, at least one cell in contact both with a lumen and with the medium outside the microtissue, and at least one cell surrounded only by cells.

Advantageously, the method according to the invention makes it possible to produce the liver microtissue according to the invention on a large scale so as to form a set of liver microtissues that can form up to 20% of the mass of the liver of the patient to be treated in less than 50 days, preferably in less than 40 days, in particular in less than 35 days.

In a preferred variant, the method according to the invention comprises at least one re-encapsulation of the liver microtissue after step (c), that is to say, at least two encapsulation cycles. Preferably, each encapsulation cycle corresponds to one pass. In this variant of the method (at least one re-encapsulation of the cells after step (c)), the number of cell divisions of the entire method (for all the passes) is at least 3, 4, 5, 6, 7, 8, 9, 10, 11, 12, 13, 14, 15, 20, 25, 30 cell division cycles.

In a method according to the invention, there may be several re-encapsulations, preferably between 1 and 100, in particular between 1 and 10 re-encapsulation(s).

Each re-encapsulation may comprise:
a step that consists in dissociating the microcompartment or the series of microcompartments to obtain liver microtissue; the removal of the outer hydrogel layer can be carried out in particular by hydrolysis, dissolution, piercing and/or rupture by any means that is biocompatible, that is to say, non-toxic for the cells. For example, removal can be achieved using phosphate buffered saline, a divalent ion chelator, an enzyme such as alginate lyase if the hydrogel comprises alginate, and/or laser microdissection, and
a step of re-encapsulation of all or part of the liver microtissue in a hydrogel capsule. Re-encapsulation is a suitable means for increasing the cellular amplification obtained from the pluripotent stage, and reducing the risks of mutation.

Re-encapsulation consists in eliminating the outer hydrogel layer, preferably in resuspending, in a partially or totally dissociated manner, the cells that were in the form of cysts in the microcompartments and re-implementing the steps of the method.

According to one embodiment, the re-encapsulation comprises the following steps:
(i) removing the outer hydrogel layer,
(ii) resuspending the liver microtissue that was contained in the microcompartment
(iii) encapsulating the liver microtissue in a layer of hydrogel so as to form a microcompartment of ovoid, cylindrical, spheroid or spherical or substantially ovoid, cylindrical, spheroid or spherical or ellipsoidal shape, comprising an outer hydrogel layer defining an inner part, the smallest radius or the average radius of said inner part being at least 300 μm;
(iv) cultivating the microcompartments obtained in a culture medium
(vii) optionally, recovering the cell microcompartments obtained.

Compartmentalization in microcompartments makes it possible to eliminate the microcompartments containing more mutated cells than the other capsules. Even if the mutated cells grow rapidly, they will reach capsular confluence, which will contain their multiplication. Compartmentalization also makes it possible not to contaminate the entire cell population, and also to eliminate the capsules containing mutant cells, at any time, in particular before a re-encapsulation step. This sorting can be done either by online analysis, or by eliminating the capsules filled more quickly than the others, for example.

In one embodiment, at least one of the steps (preferably all the steps) is carried out at a temperature adapted to the survival of the cells, comprised between 4 and 42° C. The temperature during cell proliferation should preferably be between 32 and 37° C. to avoid triggering mutations by lowering the performance of repair enzymes. Also, preferably, the temperature should be low (ideally about 4° C.) in order to manage the stress of the cells in step (c).

At any time, the method according to the invention may comprise a step consisting in verifying the phenotype of the cells contained in the microcompartment. This verification can be carried out by identifying the expression by at least some of the cells contained in the microcompartment of specific markers of the desired phenotype, chosen from:
the expression of alpha-fetoprotein, albumin and cytokeratin 19 to characterize hepatoblasts
the expression of alpha-fetoprotein and albumin and the absence of expression of cytokeratin 19 to characterize immature hepatocytes
the expression of albumin and the absence of expression of alpha-fetoprotein or cytokeratin 19 to characterize mature hepatocytes
the expression of cytokeratin and the absence of expression of albumin and alpha-fetoprotein to characterize cholangiocytes
the expression of CD31 to characterize endothelial cells
the expression of CD90 and CD73 to characterize mesenchymal stem cells.

The cell microcompartments obtained according to the methods of the invention can then be frozen before any use. The freezing is preferably carried out at a temperature between −190° C. and −80° C. Thawing can be carried out by immersing the sealed freezing vehicle (screw-on bulb or plastic bag) in a bath of lukewarm water (preferably 37 degrees) so that the cells thaw fairly quickly. Before their use, the microcompartments according to the invention can be maintained at more than 4° C. for a limited time before their use, preferably between 4° C. and 38° C.

The invention especially promotes amplification with a high rate of expansion, which consequently reduces the culture time and the number of divisions to obtain a very large number of functional lymphocytes.

### EXAMPLE

### Example 1: Differentiation Protocol

The liver microtissue according to the invention can be obtained from any known differentiation method.

To demonstrate this, the inventors have adapted 2 protocols known from the prior art. Protocol A described by Raggi et al., Stem cell Report 2022 and protocol B adapted from Mallanna and Duncan, Curr Protoc Cell Bil, 2014 and Raggi et al., Stem Cell Reports, 2022.

Materials and Method

The iPSCs were cultured on T75 flasks coated with vitronectin and were passaged regularly using the dissociation reagent ReLeSR (Stem Cell Technologies). All experiments were performed with iPSCs between passes 20 and 26.

The encapsulation of the iPSCs was carried out in alginate microcapsules of similar size between the two protocols A and B and in the presence of extracellular matrix.

For Protocol A:
Minimum diameter on the long axis=220 μm
Maximum diameter on the long axis=550 μm
Average diameter on the long axis=415 μm

For protocol B:
Minimum diameter on the short axis=200 μm
Maximum diameter on the short axis=385 μm
Average diameter on the short axis=295 μm

The iPSCs were first detached with accutase in small groups of 3 to 5 cells and resuspended at a concentration of 0.8 E6 cells/mL in a mix composed of 50% Matrigel and 50% mTESR1 medium containing 10 μM of Rhock inhibitor (Y-27632) and encapsulated by adapting the method and the microfluidic device described in Alessandri et al., 2016 (“A 3D printed microfluidic device for production of functionalized hydrogel microcapsules for culture and differentiation of human Neuronal Stem Cells (hNSC),” Lab on a Chip, 2016, vol. 16, no. 9, pp. 1593-1604). The encapsulated cells were resuspended in an mTESR1 medium containing 10 μM of Rhock inhibitor in a proportion of 0.2 mL of capsule for 1 mL of total medium. The cells were allowed to form cysts in the capsules for 96 h under stirring conditions, using a 30 mL ABLE bioreactor (Reprocell), at 37° C. and 5% C02 with a change of medium every 24 h in mTESR1 medium.

Differentiation was initiated 96 h after encapsulation by passing through RPMI medium, containing 1 mM Ca2+, 1% KnockOut replacement serum (KOSR), B27 without insulin, 100 ng/mL Activin A and 3 μM CHIR-99021 for 2 days.

During the following 3 days, the medium was replaced by RPMI medium, containing 1 mM Ca2+, 1% KOSR, B27 without insulin and 100 ng/mL Activin A.

Then, during the following 5 days, the medium was replaced by an RPMI medium, containing for protocol A: 1 mM Ca2+, 1% KOSR, B27 without insulin, supplemented with 20 ng/mL of BMP-4, 5 ng/mL of bFGF, 1 μM of A83-01 (TGF beta pathway inhibitor) and 4 μM of IWP-2 (Wnt channel inhibitor).

For protocol B: 1 mM Ca2+, 1% KOSR, B27 without insulin, supplemented with 20 ng/mL BMP-4 and 5 ng/mL bFGF. From day 11 to day 15, the medium was composed of RPMI medium, containing 1 mM Ca2+, 2% KOSR, B27 with insulin, supplemented with 20 ng/mL HGF, 3 μM CHIR-99021, 5 ng/mL bFGF and 20 ng/mL BMP-4.

On day 16, the medium was changed to HCM medium without EGF (Lonza), supplemented with 20 ng/mL HGF, 3 μM CHIR-99021, 5 ng/mL bFGF, 20 ng/mL BMP-4, 20 ng/mL Oncostatin M, 10 μM Dexamethasone and 1% KOSR.

From day 20 and for the following 5 days, the medium was composed of HCM medium plus EGF, supplemented with 20 ng/mL Oncostatin M, 10 μM Dexamethasone and 1% KOSR.

From the 25th day, the medium was composed of HCM medium plus EGF, 10 μM Dexamethasone and 1% KOSR.

Gene Expression Analysis

Gene expression analysis was performed by RT-Q-PCR.

qPCR was performed on a Roche LightCycler® 480 instrument. The expression of each gene was normalized by the expression of the reference genes YWHAZ, NONO and VCP. Data are expressed as 2\hat{ }delta Ct, where delta Ct=Ct of gene−average Ct of the reference genes.

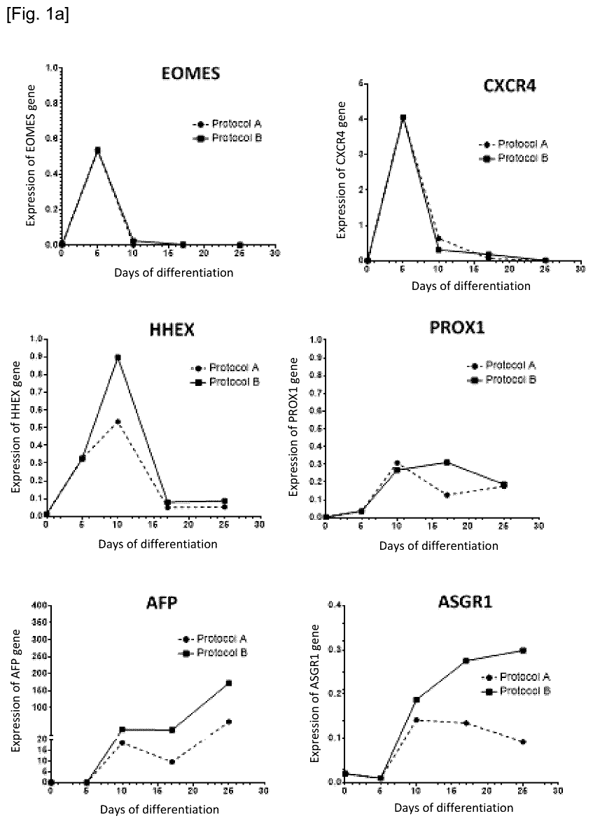

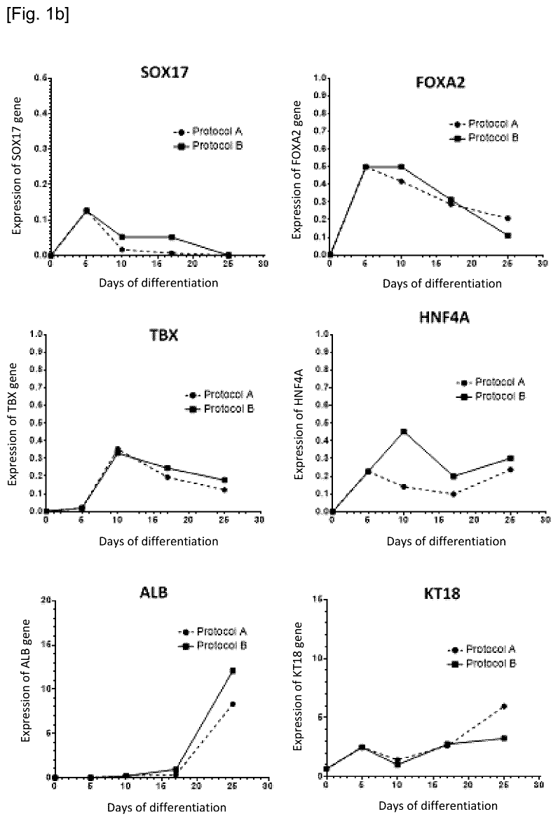

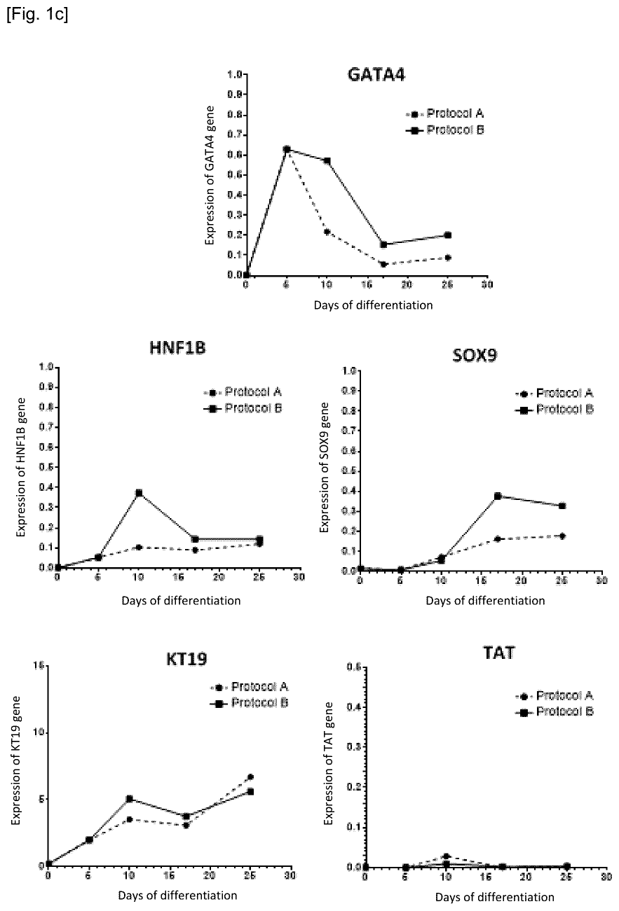

The results are shown in FIGS. 1a, 1b and 1c. These results show that the liver microtissue according to the invention can be obtained with any suitable differentiation method.

### Example 2: Differentiation into Hepatocytes within the Microcompartment According to the Invention vs. In Two Dimensions

The encapsulation of the iPSCs was carried out in alginate microcapsules with the characteristics below:
Minimum diameter on the long axis=450 μm
Maximum diameter on the long axis=685 μm
Average diameter on the long axis=575 μm
Minimum diameter on the short axis=313 μm
Maximum diameter on the short axis=546 μm
Average diameter on the short axis=423 μm

For the encapsulation of the iPSCs in alginate microcapsules of approximately 575 μm in average diameter and in the absence of matrix, the iPSCs were first detached with accutase in small groups of 3 to 5 cells and resuspended at a concentration of 10 E6 cells/mL in an mTESR1 medium containing 10 μM of Rhock inhibitor (Y-27632) and encapsulated by adapting the method and the microfluidic device described in Alessandri et al., 2016 (“A 3D printed microfluidic device for production of functionalized hydrogel microcapsules for culture and differentiation of human Neuronal Stem Cells (hNSC),” Lab on a Chip, 2016, vol. 16, no. 9, Pp. 1593-1604). The encapsulated cells were resuspended in an mTESR1 medium containing 10 μM of Rhock inhibitor at 0.5 E6 cells/mL, the proportion of capsules relative to the medium not exceeding 20%. The cells were allowed to agglutinate in the capsules for 24 h under stirring conditions, using a 30 mL ABLE bioreactor (Reprocell), at 37° C. and 5% CO2.

Differentiation was initiated 24 h after encapsulation by passing through RPMI medium, containing 1 mM Ca2+, 1% KnockOut replacement serum (KOSR), B27 without insulin, 100 ng/mL Activin A and 3 μM CHIR-99021 for 2 days.

During the following 3 days, the medium was replaced by RPMI medium, containing 1 mM Ca2+, 1% KOSR, B27 without insulin and 100 ng/mL Activin A.

Then, for the next 5 days, the medium was replaced with RPMI medium, containing 1 mM Ca2+, 1% KOSR, B27 without insulin, supplemented with 20 ng/mL BMP-4 and 5 ng/mL bFGF. From day 11 to day 15, the medium was composed of RPMI medium, containing 1 mM Ca2+, 2% KOSR, B27 with insulin, supplemented with 20 ng/mL HGF, 3 μM CHIR-99021, 5 ng/mL bFGF and 20 ng/mL BMP-4.

On day 16, the medium was changed to HCM medium without EGF (Lonza), supplemented with 20 ng/mL HGF, 3 μM CHIR-99021, 5 ng/mL bFGF, 20 ng/mL BMP-4, 20 ng/mL Oncostatin M, 10 μM Dexamethasone and 1% KOSR.

From day 20 and for the following 5 days, the medium was composed of HCM medium plus EGF, supplemented with 20 ng/mL Oncostatin M, 10 μM Dexamethasone and 1% KOSR.

From the 25th day, the medium was composed of HCM medium plus EGF, 10 μM Dexamethasone and 1% KOSR.

The medium was changed daily from day 1 to day 25, and every other day from day 25.

2D Hepatocyte Differentiation

The iPSCs were dissociated into individual cells using accutase and placed on 6-well plates coated with Matrigel in mTESR1 medium containing 10 μM of Rhock inhibitor at a density of 1×105 cells/cm2. Differentiation was initiated 24 h after plating using the same differentiation protocol and medium composition as for 3D capsules.

Gene Expression Analysis

Gene expression analysis was performed by RT-Q-PCR.

qPCR was performed on a Roche LightCycler® 480 instrument. The expression of each gene was normalized by the expression of the reference genes YWHAZ, NONO and VCP. Data are expressed as 2\hat{ }delta Ct, where delta Ct=Ct of gene−average Ct of the reference genes.

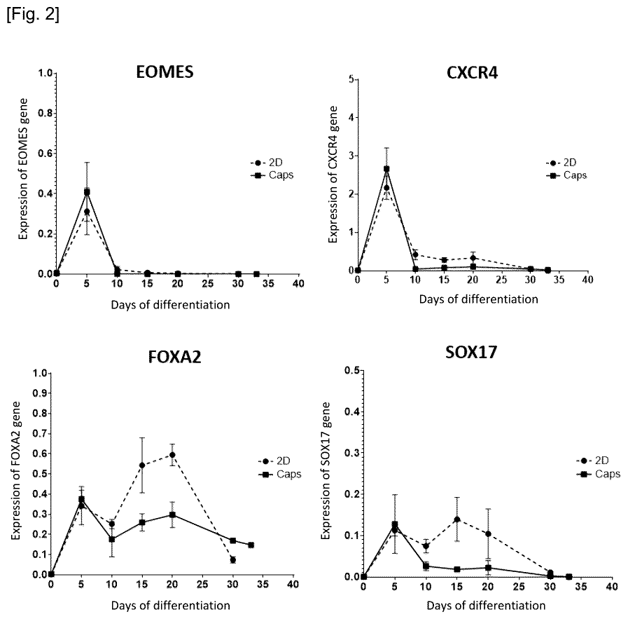

The comparison of gene expression between hepatocytes obtained in two dimensions versus three dimensions is shown in FIG. 2 in order to verify the induction of the endoderm during differentiation. The expression of the EOMES, CXCR4, FOXA2 and SOX17 genes shows that the differentiation indeed goes through a transitory stage of endodermal differentiation toward the definitive endoderm, which passage is expected to generate liver cells from pluripotent cells.

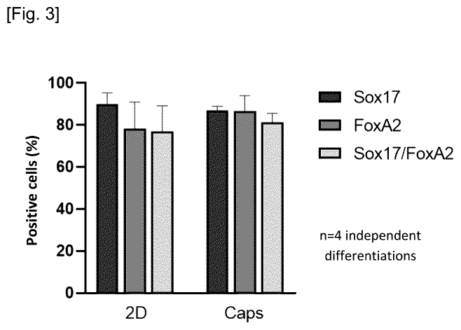

The analysis of the protein expression of endoderm markers was performed at day 5 and is shown in FIG. 3. These results show that the differentiation progresses according to a sequence in accordance with the expectations of those skilled in the art, that is to say, via the definitive endoderm and toward the liver bud.

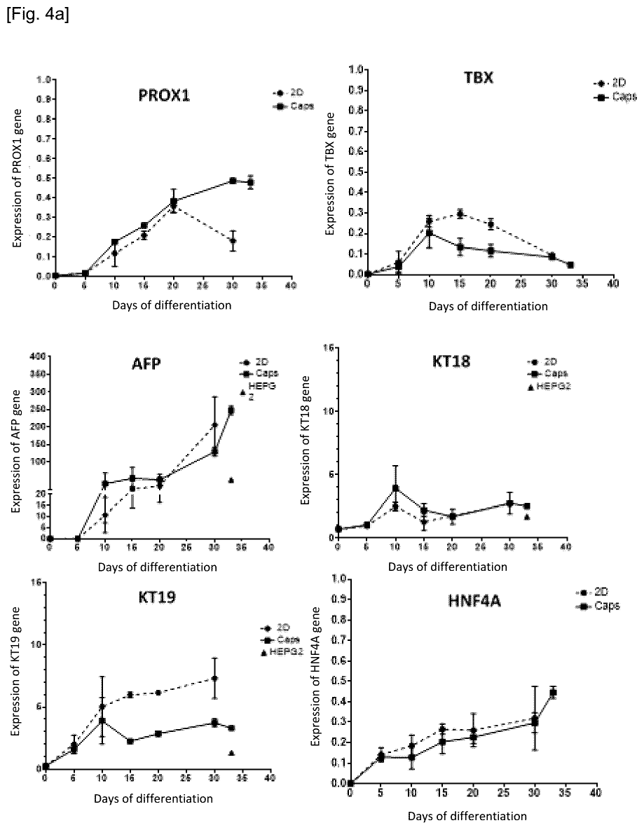

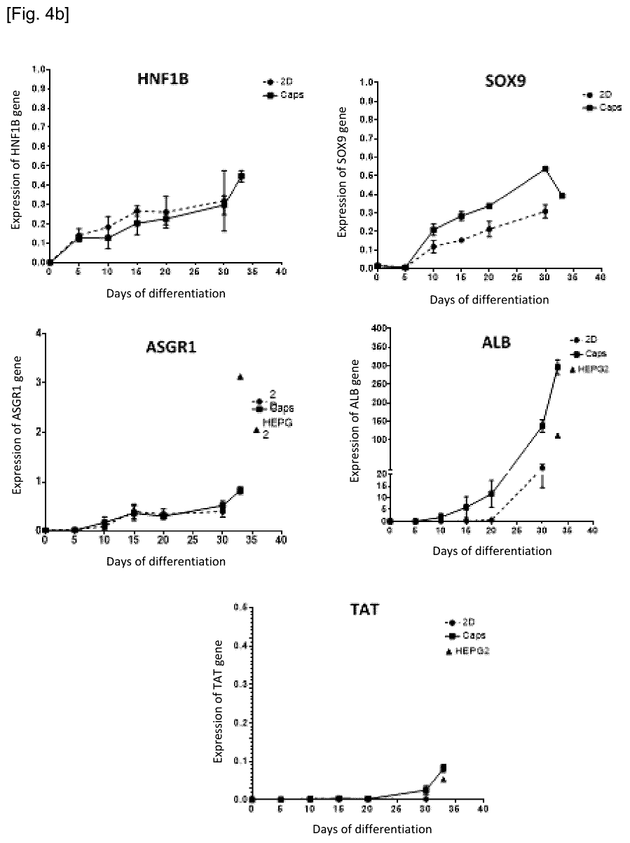

An analysis of gene expression during differentiation protocols is shown in FIGS. 4a and 4b. These results show that the differentiation progresses toward a composition that is consistent with the target of the cellular composition of the liver, in particular hepatocytes.

Measurement of the Rate of Expansion:

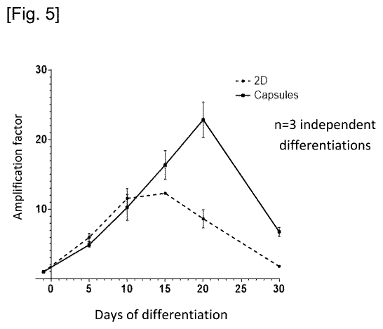

The rate of expansion is measured by taking the ratio of the number of cells counted on day X of the culture divided by the number of cells at the start of the culture (day of encapsulation or day of placement in culture). The rate of expansion or amplification factor was measured during differentiation. The tracking of the rate of expansion during the differentiation process is shown in FIG. 5. A rate of expansion of at least 15 times, 20 days after the start of differentiation is found.

Assessment of Albumin and Urea Production

To assess albumin and urea production, the conditioned medium was sampled periodically 24 hours (±2 h) after the medium change and stored at −80° C. The levels of albumin and urea secreted into the medium were measured using the ELISA kit for human albumin (Invitrogen) and the Quantichrom urea assay kit (Gentaur), respectively, according to the manufacturer's instructions. The quantity of molecules secreted in 24 h was then normalized by the number of cells according to the count carried out after dissociation for each time point.

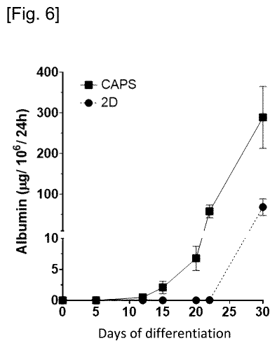

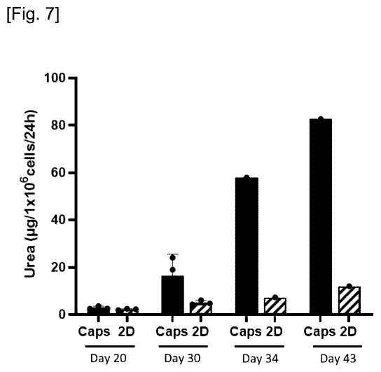

The results are shown in FIGS. 6 and 7. It is found that albumin secretion and urea production are greater in the microcompartments according to the invention.

Measurement of CYP3A4 Activity:

Cyp3A4 activity was performed using the P450-Glo™ CYP3A4 assay with Luciferin-IPA (Promega) according to the manufacturer's instructions. Briefly, a sample of the 3D capsules containing the microtissues was decapsulated and the number of cells in a given volume of capsules containing the microtissues was determined. The decapsulated microtissues corresponding to the 1E5 cells, or to the 1E5 cells that were differentiated in 2D, were mixed with 50 μl of the substrate proluciferin P450-Glo 3 μM in a 96-well plate with a round bottom and incubated for 3 h at 37° C., 5% CO2. For each condition, 5 repetitions were performed, cell culture medium alone, and iPSCs were used as negative controls. Subsequently, 25 μl of the culture medium from each well was transferred to an opaque white 96-well luminometer plate and mixed with 25 μl of luciferin detection reagent. The plate was incubated for 20 minutes at room temperature and the luminescence was read using the Spectramax i3x microplate reader (Molecular devices) with an integration time of 1 second per well. The net signal was calculated by subtracting background luminescence values from control wells without cells.

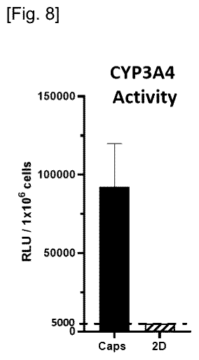

The results are shown in FIG. 8. A significantly greater activity of CYP3A4 of the cells encapsulated in the microcompartment according to the invention is observed.

### Example 3: Morphological Study of the Microcompartment According to the Invention

The objective of this example is to characterize the morphology of cells within the microcompartment during differentiation.

Histology

Histological analysis was performed as a paid service at Novotec Laboratories.

Liver microtissue samples were fixed with AFA fixative for 24 h at room temperature, washed with PBS and pre-embedded in histogel. After dehydration in successive baths of ethanol, acetone and xylene, the samples were embedded in paraffin and sectioned with a microtome to a thickness of 5 μm.

Hematoxylin-Eosin-Saffron (HES) Staining

After paraffin removal, sections were stained with Harris's hematoxylin and eosin G. After dehydration, sections were stained with Saffron and mounted with Entellan. The cellular cytoplasm is stained in pink, the nuclei in violet-blue and the extracellular matrix in yellow-pink.

PAS (Periodic-Acid-Schiff) Staining

After paraffin removal, sections were pretreated with 1% periodic acid, followed by sequential staining with Schiff s reagent and Mayer's hematoxylin. The glycogen is stained pink and the nuclei blue-violet.

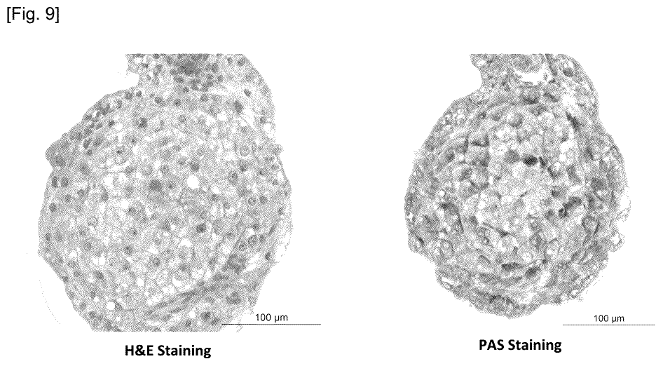

The images resulting from the staining are shown in FIG. 9. The characteristic cubic shape of hepatocytes can be clearly seen, as well as the presence of glycogen granules within the microcompartment.

Immunofluorescence on 3D Microtissues

The microtissues were fixed inside the capsules in a solution of 4% paraformaldehyde in PBS with calcium for 1 hour at room temperature. After being rinsed with Ca2+-free PBS to remove the capsules, they were permeabilized in 1% Triton X-100 for 30-60 minutes at room temperature. The microtissues were incubated with the primary antibody solution for 72 h at 4° C. with stirring. After washing with PBS, they were incubated with a solution of marked secondary antibodies (Alexa Fluor, Life Technologies) and DAPI overnight at 4° C. with stirring and protected from light.

For structural imaging by phalloidin-DAPI, the staining was done while preserving the capsules (the decapsulation step before permeabilization was skipped, the washings were done with PBS containing calcium). The microtissues were incubated with DAPI and Phalloidin for 72 h at 4° C. with stirring.

After washing with PBS, the cells were mounted on a slide with a spacing of 0.5 mm. Images were acquired on a confocal microscope (SP5, Leica).

TABLE 3

| Antibody | Company | Ig Species | Dilution |
|---|---|---|---|
| AFP | DAKO | Rabbit | 1:2000 |
| ALB | Bethyl | Goat | 1:400 |
| CK19 | DAKO | Mouse | 1:100 |

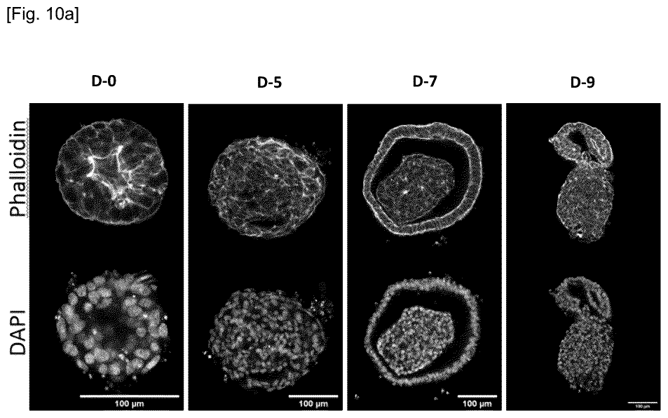

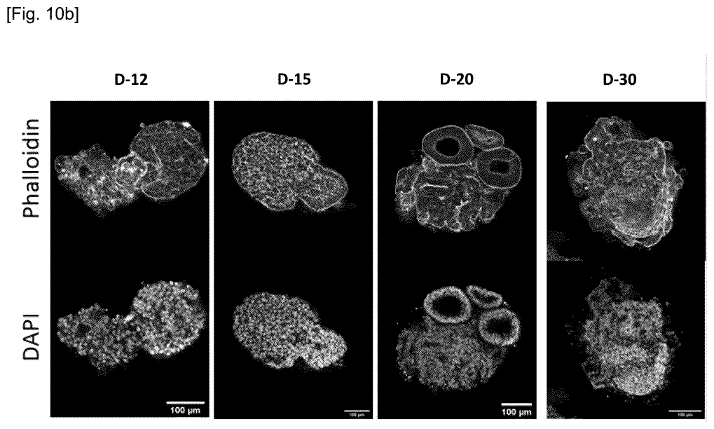

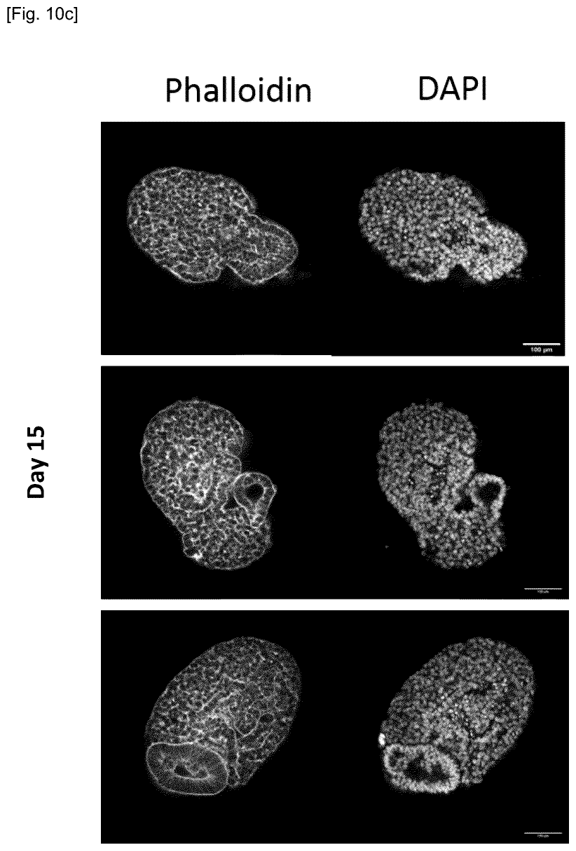

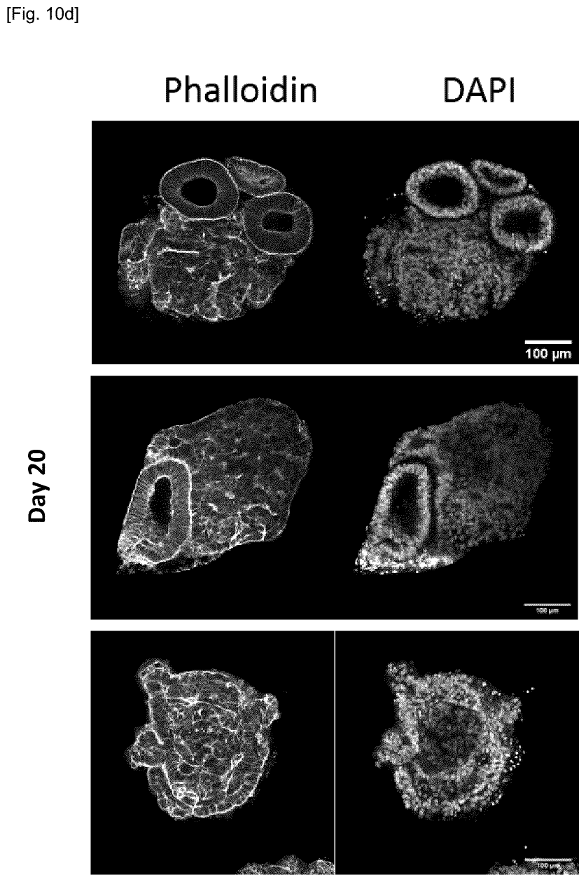

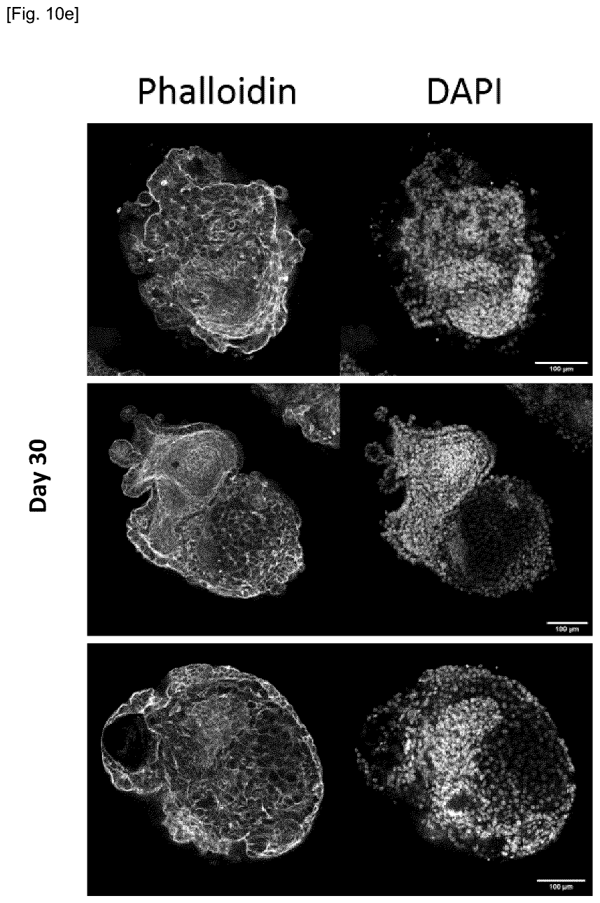

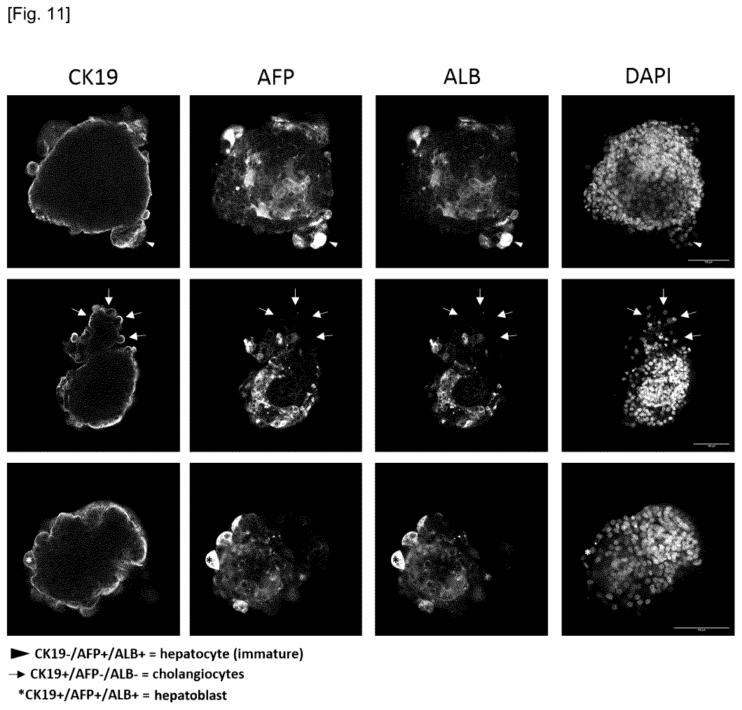

The evolution of the microcompartment's morphology was carried out for 30 days during the differentiation. These results are shown in FIGS. 10a to 10e. The presence of specific structures can be observed, such as the formation of a structure similar to that of a liver bud on day 7 and a cellular organization characteristic of the liver microtissue according to the invention. Indeed, one can see at least one lumen, at least one cell in contact both with a lumen and with the medium outside the microtissue, and at least one cell surrounded only by cells. In addition to the study of the morphology, the inventors were able to characterize the presence of at least 3 different phenotypes of liver cells within the microcompartment, 30 days after the start of differentiation (FIG. 11).

We claim:

1. A three-dimensional liver microtissue, the largest dimension of which is between 500 and 700 μm, expressing CYP3A4 monooxygenase with an activity of at least 75,000 RLU per million cells and producing at least 18 μg of urea per million cells per 24 hours.

2. The three-dimensional liver microtissue according of claim 1, wherein the microtissue comprises between 50 and 99% liver cells.

3. The three-dimensional liver microtissue according to claim 2, wherein the microtissue contains cells which are a progeny of induced pluripotent stem cells.

4. The three-dimensional liver microtissue of claim 1, wherein the microtissue contains cells which are a progeny of induced pluripotent stem cells encapsulated in a single closed three-dimensional microcompartment.

5. The three-dimensional liver microtissue of claim 2, wherein the liver cells secrete at least 75 kg of albumin per million cells per 24 hours.

6. The three-dimensional liver microtissue of claim 1, wherein the microtissue comprises liver cells being differentiated from induced pluripotent stem cells and the liver cells secrete at least 75 kg of albumin per million cells per 24 hours at least 20 days after the start of differentiation.

7. The three-dimensional liver microtissue of claim 1 comprising at least 3 different liver cell phenotypes, wherein all the cells of the microtissue have been produced from induced pluripotent stem cells encapsulated in a single closed three-dimensional microcompartment.

8. The three-dimensional liver microtissue of claim 1, wherein the liver microtissue comprises immature hepatocytes, mature hepatocytes and cholangiocytes.

9. The three-dimensional liver microtissue of claim 1, wherein the liver microtissue comprises at least:
immature hepatocytes expressing alpha-fetoprotein and albumin and not expressing cytokeratin 19;
mature hepatocytes expressing albumin and not expressing expression alpha-fetoprotein and cytokeratin 19; and
cholangiocytes expressing cytokeratin 19 and not expressing albumin and alpha-fetoprotein.

10. The three-dimensional liver microtissue of claim 1, wherein the liver microtissue comprises cells expressing CD73 and CD90.

11. The three-dimensional liver microtissue of claim 10, wherein the cells expressing CD73 and CD90 are mesenchymal stem cells.

12. The three-dimensional liver microtissue of claim 1, wherein the liver microtissue comprises at least liver cells, of which between 20 and 60% of the cells of the liver cells are cells expressing cytokeratin 19.

13. The three-dimensional liver microtissue of claim 1, wherein all cells of the microtissue have been produced by differentiation of at least one cyst of induced pluripotent stem cells encapsulated in a single closed three-dimensional microcompartment.

14. The three-dimensional liver microtissue of claim 1, comprising:
at least one lumen,
at least one cell in contact both with a lumen and with the medium outside the microtissue, and
at least one cell surrounded only by cells.

15. The three-dimensional liver microtissue of claim 1, wherein the microtissue has an ellipsoidal shape.

16. The three-dimensional liver microtissue of claim 2, wherein the liver cells are polarized.

17. The three-dimensional liver microtissue of claim 1, wherein the microtissue comprises between 50% and 99% liver cells, of which between 20% and 60% of the liver cells are cells expressing cytokeratin 19, and between 1% and 20% cells expressing CD73 and CD90.

18. The three-dimensional liver microtissue of claim 1, wherein the microtissue has a diameter or the smallest dimension of between 100 μm and 300 μm.

19. The three-dimensional liver microtissue of claim 1, wherein the microtissue has the largest dimension of between 500 μm and 700 μm.

20. The three-dimensional liver microtissue of claim 1, wherein the microtissue comprises between 300 and 14,000 cells.

21. The three-dimensional liver microtissue of claim 1, wherein the microtissue comprises at least one bile duct.

22. The three-dimensional liver microtissue of claim 1, wherein the microtissue comprises at least one glycogen granule.

23. The three-dimensional liver microtissue of claim 2, wherein the liver cells include one or more of the following: mature hepatocytes, immature hepatocytes, hepatoblasts, cholangiocytes, or any mixture thereof.

24. A closed three-dimensional cell microcompartment comprising an outer hydrogel layer defining an inner part, said inner part comprising at least one liver microtissue of claim 1.

25. The closed three-dimensional cell microcompartment of claim 24, wherein the microcompartment is harvested 20 days after the encapsulation of 1 to 200 induced pluripotent stem cells in the inner part defined by the outer hydrogel layer.

26. The closed three-dimensional cell microcompartment of claim 24, wherein the microcompartment exhibits a rate of expansion of at least 15 times, 20 days after the start of differentiation.

27. The closed three-dimensional cell microcompartment of claim 24, wherein the thickness of the outer layer is variable and between 20 and 60 μm.

28. The closed three-dimensional cell microcompartment of claim 24, wherein the outer layer comprises alginate.

29. The closed three-dimensional cell microcompartment of claim 24, wherein the microcompartment comprises extracellular matrix elements or a natural or synthetic extracellular matrix in the inner part between the outer layer and the liver microtissue.

30. The closed three-dimensional cell microcompartment of claim 24, wherein the microcompartment has a diameter or the smallest dimension of between 300 μm and 400 μm.

31. The closed three-dimensional cell microcompartment of claim 24, wherein the microcompartment has the largest dimension of between 400 μm and 600 μm.

32. A set or series of microcompartments comprising at least two three-dimensional cell microcompartments, wherein at least one microcompartment is a microcompartment according to claim 24.

33. The set of microcompartments of claim 32, wherein the microcompartments are placed in a culture medium in a bioreactor.

34. A drug comprising the three-dimensional liver microtissue of claim 1 or a closed three-dimensional cell microcompartment comprising an outer hydrogel layer defining an inner part, said inner part comprising the microtissue.

35. A method of treating symptoms associated with liver failure in a subject, the method comprising administering to the subject the three-dimensional liver microtissue of claim 1 or a closed three-dimensional cell microcompartment comprising an outer hydrogel layer defining an inner part, said inner part comprising the microtissue.

36. The method of claim 35, wherein the liver failure is acute, chronic or acute-on-chronic liver failure.

37. A method of treating or preventing a metabolic disease of the liver in a subject, the method comprising administering to the subject the three-dimensional liver microtissue of claim 1, or a closed three-dimensional cell microcompartment comprising an outer hydrogel layer defining an inner part, said inner part comprising the microtissue.

38. A method of treating or preventing in a subject hepatic fibrosis and cirrhosis, steatosis, non-alcoholic steatosis, hepatitis, a disease linked to secretion of factor VIII and factor IX and VWF, Wilson's disease, or hereditary hemochromatosis, the method comprising administering to the subject the microtissue of claim 1, or an closed three-dimensional cell microcompartment comprising an outer hydrogel layer defining an inner part, said inner part comprising the microtissue.

39. A method for preparing the microcompartment of claim 24, the method comprising the steps consisting in:
a) producing a closed three-dimensional cell microcompartment comprising induced pluripotent stem cells inside an outer hydrogel layer, and optionally comprising extracellular matrix elements or a natural or synthetic extracellular matrix, and
b) inducing cell differentiation within the cell microcompartment, so as to obtain a microcompartment comprising liver cells.

40. The method of claim 39, wherein step b) lasts at least 20 days.

41. The method of claim 39, wherein in step a) between 40 and 150 induced pluripotent stem cells are present in the microcompartment.

42. A method for preparing the liver microtissue of claim 1, wherein the method comprises:
preparing a closed three-dimensional cell microcompartment comprising an outer hydrogel layer defining an inner part, said inner part comprising the liver microtissue, and
eliminating the outer hydrogel layer to recover the liver microtissue.

43. A method for transplanting a liver tissue into a subject, the method comprising transplanting to the subject one or more of the three-dimensional liver microtissues of claim 1 and/or a closed three-dimensional cell microcompartment comprising an outer hydrogel layer defining an inner part, said inner part comprising the microtissue.

44. The method of claim 43, wherein the subject is at risk of liver failure and/or has been diagnosed with a metabolic disease of the liver.
